(19) United States
(12) Patent Application Publication
(10) Pub. No.: US 2025/0054578 A1
(43) Pub. Date: Feb. 13, 2025

(54) SYSTEMS AND METHODS FOR IDENTIFYING MICROBIAL SPECIES AND IMPROVING HEALTH

(71) Applicant: Sapient Bioanalytics, LLC, San Diego, CA (US)

(72) Inventors: Tao Long, San Diego, CA (US); Igor Segota, San Marcos, CA (US); Mohit Jain, La Jolla, CA (US)

(21) Appl. No.: 18/720,618

(22) PCT Filed: Dec. 15, 2022
(86) PCT No.: PCT/US2022/052987

§ 371 Date: Jun. 14, 2024

Related U.S. Application Data
(60) Provisional application No. 63/290,472, filed on Dec. 16, 2021.

(51) Int. Cl. G16B 30/00; G16B 45/00; G16B 50/10

(52) U.S. Cl. CPC G16B 30/00; G16B 45/00; G16B 50/10

(57) ABSTRACT

The present disclosure relates generally to systems and methods for identifying and qualifying microbial species, such as those isolated from a human individual. Once the individual is identified as be deficient in certain microbial species, care can be given accordingly to increase the microbial species and thus improve health of the individual.

### CROSS REFERENCE TO RELATED APPLICATIONS

This application claims the benefit under 35 U.S.C. § 119(e) of U.S. Provisional Application Ser. No. 63/290,472, filed Dec. 16, 2021, the content of which is incorporated by reference in its entirety into the present disclosure.

### BACKGROUND

The role of the gut microbiome in human health and disease is rapidly evolving, with an increasing number of studies linking the microbiome with fundamental physiologic processes such as nutrition, drug response, aging, as well as pathophysiologic states including metabolic, cardiovascular, autoimmune, neurological, and oncologic diseases¹⁻³. To date, study of the human microbiome across diverse populations has largely utilized 16S amplicon sequencing, in which the hypervariable regions of bacterial 16S rRNA genes are PCR amplified and sequenced. Amplicon sequencing data are processed using common pipelines, such as QIIME2⁴, DADA2⁵, and Mothur⁶, with taxonomic assignment performed through computational reconstruction of the phylogenic tree, using databases including Greengenes⁷, SILVA⁸, or RDP⁹. 16S sequencing has therefore enabled evaluation of changes in overall microbial diversity across human phenotypes, with identification of key components of the human microbiome at the level of family or genus^{10,11} While serving to establish the overall importance of the gut microbiome in human health and disease, 16S amplicon sequencing has been limited in its ability to discriminate microbes at a finer species level. Upon inspecting the two most widely used 16S databases, Greengenes and SILVA, the instant inventors found they disagreed in the species assignment for more than half of identical, full-length 16S sequences (FIG. 7). Consecutively, 16S sequencing has been limited in mapping microbial sequences to the species level.

Causal investigation of specific microbes for both disease risk and therapeutic potential necessitates higher resolution identification of microbes within the gut. Fine mapping of microbial species has largely been enabled through newer whole-genome shotgun sequencing (WGS) approaches of gut microbiota. WGS typically necessitates deep sequencing coverage and extensive computational effort, precluding routine application on a population scale across tens of thousands of individuals.

In parallel with advances in the study of the microbiome, the number of gut microbial strains isolated and fully sequenced from humans has continued to exponentially increase.

### SUMMARY

The present disclosure relates generally to systems and methods for identifying and qualifying microbial species, such as those isolated from a human individual. Once the individual is identified as be deficient in certain microbial species, care can be given accordingly to increase the microbial species and thus improve health of the individual.

One embodiment of the present disclosure provides a method for improving the health of a human subject, comprising: obtaining a biological sample from the human subject, wherein the biological sample comprises microbial species residing in the human subject; sequencing hypervariable regions of 16S rRNA genes in bacterial genomes in the biological sample, and counting the copy number of each hypervariable region in the 16S rRNA genes in each genome; comparing the hypervariable region sequences and copy numbers thereof in the 16S rRNA genes to a database comprising hypervariable region sequences and copy numbers thereof in the 16S rRNA genes of different bacterial species, to identify the bacterial species of the 16S rRNA genes; comparing the identified bacterial species to common bacterial species found in human subjects, to identify bacterial species deficient in the human subject; and administering to the human subject a dietary supplement supplying, or promoting the growth of, the deficient bacterial species.

In some embodiments, the common bacterial species comprise at least 5 selected from the group consisting of: Eubacterium rectale, Anaerostipes hadrus, Blautia faecis, Blautia obeum, Blautia obeum/wexlerae, Dorea longicatena, Fusicatenibacter saccharivorans, Roseburia inulinivorans, Oscillibacter sp., Romboutsia timonensis, Faecalibacterium prausnitzii, and Gemmiger formicilis.

In some embodiments, the common bacterial species comprise at least 6, 7, 8, 9, 10, 11 or 12 species selected from the group.

In some embodiments, the dietary supplement supplies the deficient bacterial species.

Also provided is a method for identifying the bacterial species of a genomic DNA sample, comprising: sequencing hypervariable regions of 16S rRNA genes in the genomic DNA sample, and counting the copy number of each hypervariable region in the 16S rRNA genes in the genomic DNA sample; and comparing the hypervariable region sequences and copy numbers thereof in the 16S rRNA genes to a database comprising hypervariable region sequences and copy numbers thereof in 16S rRNA genes of different bacterial species, to identify the bacterial species of the 16S rRNA genes.

In some embodiments, the method further comprises pre-processing the hypervariable region sequences, wherein the pre-processing comprises one or more of de-noising, un-trimming, and abundance estimation.

In some embodiments, the method further comprises trimming the sequences to a predetermined length, prior to un-trimming, which comprises concatenating the trimmed sequences.

In some embodiments, the method further comprises aligning the sequences to the database.

In some embodiments, the database is prepared by a method comprising: acquiring genome sequences from a plurality of bacterial species and strains; extracting 16S rRNA sequences from the genome sequences; eliminating sequences that are not within hypervariable regions of 16S rRNA genes; counting sequence variants and copy numbers of unique hypervariable regions; and identifying a list of 16S rRNA gene sequences from the bacterial species and strains.

In some embodiments, preparation of the database further comprises cleaning the 16S rRNA gene sequences in the list by removing outliers.

In some embodiments, preparation of the database further comprises cleaning the 16S rRNA gene sequences in the list by removing multi-annotated ones.

Still further provided is a method for preparing a database of 16S rRNA genes useful for identifying bacterial species, comprising: acquiring genome sequences from a plurality of bacterial species and strains; extracting 16S rRNA sequences from the genome sequences; eliminating sequences that are not within hypervariable regions of 16S rRNA genes; counting sequence variants and copy numbers of unique hypervariable regions; and identifying a list of 16S rRNA gene sequences from the bacterial species and strains.

In some embodiments, preparation of the database further comprises cleaning the 16S rRNA gene sequences in the list by removing outliers.

In some embodiments, preparation of the database further comprises cleaning the 16S rRNA gene sequences in the list by removing multi-annotated ones.

### BRIEF DESCRIPTION OF THE DRAWINGS

FIG. 1. Schematic and benchmarking of RExMap. A. RExMap reference database (RExMapDB) is constructed using 16S rRNA gene sequences from the NCBI Genome and RefSeq databases of Bacteria and Archaea, by extracting hypervariable region variants and the corresponding copy numbers of each unique isolate strain. B. RExMap consists of pre-processing (merging, PCR primer removal, quality control), denoising inferring exact sequence variants, aligning these sequences to the RExMapDB, and aggregating sequences belonging to the undistinguishable strains into Operational Strain Units (OSUs). C-D. Identification and abundance estimates of mock 20-microbe community and a human fecal sample with paired 16S sequencing of V3-V4 and WGS data. E. Distribution of the fraction of MetaPhlAn2 species that are captured by RExMap. F. Comparison of relative microbial abundances estimated by WGS MetaPhlAn2 and 16S RExMap for a 780-sample human gut microbiome dataset (DIABIMMUNE).

FIG. 2. The World Microbiome Database. A. Geographical distribution of samples. B and C. Distribution of human samples based on the sampling site on the human body. D. Pie-chart of samples categorized by diseases and conditions following MeSH terms. Acronyms: Fem. Urog. Sys.=Female Urogenital Diseases and Pregnancy Complications including Infant samples.

FIG. 3. RExMap analysis of 29,349 human gut microbiomes. A. Color map of the number of samples obtained from each region. List of regions with number of samples and reference studies. B. Relation between regional mean abundance and regional prevalence of 17,786 OSUs. C. Number of unique OSUs at a given regional prevalence. D. Number of OSUs contributing to the compositional abundance of each individual. E. Number of OSUs contributing to the compositional abundance of each region. The regional color legend (right) is used for C,E.

FIG. 4. Regional-specific gut microbes. A. Principal Coordinate Analysis of all samples colored by region, showing all samples colored by region along the first two principal axes. B. Top OSUs contributing to the first three principal coordinates. C. Total abundance of two Prevotella copri OSUs versus all Bacteroides OSUs across all regions.

FIG. 5. Core gut microbes across human populations. A. Distribution of abundances for each of the core OSUs across all human samples. OSUs are colored by their family rank, with different shades distinguishing OSUs within the same family B. Prevalence of core OSUs in the Twins UK dataset based on Kraken2 WGS (black) and RExMap 16S (green) data. C. Abundance distribution of core OSUs in the Twins UK dataset. D. Distribution of the total number of core OSUs. E. Distribution of the average total abundance of the core OSUs across each region. F. Composition of the core OSUs across regions. G. Colonization of core OSUs through early life. H. Number of core OSUs present in 128 vertebrate host species, plotted on a phylogenetic tree, where the color of tree leaves (host species) indicates the number of core OSUs found and internal edge colors average number of core OSUs in each clade. Inset shows the core OSU presence in non-human primates.

FIG. 6. Association between core OSUs and body mass index. Core OSUs were evaluated in three large adult populations with available BMI, age and gender phenotypes: American Gut Project (AGP, N=9,621), Guangdong Gut Microbiome Project (GGMP, N=6,748) and Twins UK (N=1,013). A. Boxplot of total core OSU abundance in four standard BMI categories. Three asterisks indicate two-sided Wilcox rank-sum tests with p-values less than 0.001 between BMI categories that validate across all three datasets. B. Effect size of individual core OSUs on BMI adjusted for age and gender. Lines indicate 95% confidence intervals. Meta-analysis (black lines) p-value is presented.

FIG. 7. Comparison of taxonomical mapping of 17,214 identical, full-length (with lengths between 1,200 and 1,600 nucleotides), 16S sequences between Greengenes v. 13.5 99% OTU and SILVA v 1.3.2 databases. Green indicates number of sequences with identical taxonomic rank assignments, red indicates different taxonomic rank assignments and gray represents number of sequences where taxonomic rank was not assigned in either Greengenes or SILVA database.

FIG. 8. The total number of sequenced genomes (left) and unique species (right) for archaea and bacteria in the NCBI Genome database as a function of time. The total number of genomes approximately follows an exponential growth with the doubling time of about 1.3 years. We obtained the number of genomes for archaea and bacteria by calculating a cumulative sum of genomes present in assembly_summary.txt files at ftp://ftp.ncbi.nlm.nih.gov/genomes/refseq/bacteria/ and ftp://ftp.ncbi.nlm.nih.gov/genomes/refseq/archaea/.

FIG. 9. A. Schematic indicating the ease of obtaining 16S variants (colored stripes) from full genome sequences (chromosome indicated in gray) and the ambiguity of mapping 16S variants back to full genomes. Results based on RExMapDB version 2020-01-20. B. Ambiguity of mapping V3-V4 sequences to full genomes. Pie-chart showing the fraction of V3-V4 sequences mapping to various number of unique genomes, indicated with 1 to 9+(9 or greater). If a single V3-V4 sequence variant is used for this mapping, about 56% of V3-V4 sequences can be traced back to a single strain. C. If the full set of unique V3-V4 sequence variants, as well as their copy numbers from each genome are used (referred to as the “OSU spectrum” in the Main text methods), about 75% of them can be mapped to an exact strain. Using multiple sequence variants can therefore increase the mapping resolution.

FIG. 10. Sequencing depth (total number of paired-end reads in the raw FASTQ files) for 16S amplicon sequencing (red) and whole-genome shotgun sequencing (blue) data in the DIABIMMUNE study for 780 matching samples.

FIG. 11. PCoA plots showing the coordinates of all samples along the first three principal axes.

FIG. 12. The composition of core OSUs for the AGP samples with self-reported different types of gut microbiome-related diseases: small intestinal bacterial overgrowth, C. diff infection, inflammatory bowel disease, recent antibiotic usage (less than a week and less than a month). The last column shows the average core OSU abundance for individuals with no self-reported diseases (labeled Healthy) from AGP.

FIG. 13 illustrates the process of pooling multiple datasets in pairs for analysis.

FIG. 14 illustrates the process of pooling multiple datasets in pairs with matching suitable datasets for analysis.

FIG. 15 is a schematic illustrating the computing components that may be used to implement various features of the embodiments described in the present disclosure.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

### Identifying Microbial Species and Improving Health

Methods for constructing a database for identifying microbial species are described, along with use of the database to conduct such identification. For an individual, the identification, optionally along with qualification, can help reveal the types and amounts of microbial species. As microbial species are related to health or future health of a person, this information can guide healthcare, such as supplementing to the individual foods (e.g., yogurt, pills) that provide the deficient microbial species.

It is hereby identified that some core microbial species are present in most of the population in the world. Also, for each demographic group, they may share their own signature microbial species groups. Therefore, in one embodiment, provided is a method for detecting microbial species in a biological sample obtained from a subject, such as a human subject. The detected microbial species are then compared to the common core microbial species among individuals having the same demographic characteristics as the subject to identify microbial species that are deficient in the subject. Subsequent to the identification, food or supplements containing the deficient microbial species can be administered to the subject for improving the health.

In accordance with one embodiment of the present disclosure, provided is a method for improving the health of a human subject. In some embodiments, the method entails identifying a human subject having deficiency of one or more core microbial species. In some embodiments, the identification entails obtaining a biological sample from the human subject, wherein the biological sample comprises microbial species residing in the human subject, sequencing hypervariable regions of 16S rRNA genes in bacterial genomes in the biological sample, and counting the copy number of the 16S rRNA genes in each genome, comparing the hypervariable region sequences and copy numbers of the 16S rRNA genes to a database comprising hypervariable region sequences and copy numbers of 16S rRNA genes of different bacterial species, to identify the bacterial species of the 16S rRNA genes, and comparing the identified bacterial species to common bacterial species found in human subjects, to identify bacterial species deficient in the human subject. Once the deficiency is identified, the human subject can be suggested or prescribed for administration of a dietary supplement supplying, or promoting the growth of, the deficient bacterial species.

The biological sample can be any sample obtained from the subject that contains microbial species residing in the gastrointestinal track. In some embodiments, the biological sample is a stool sample, an intestinal mucosal sample or a sample of intestinal contents.

In some embodiments, the biological sample is one that contains microbial species residing in a tissue selected from gut/intestinal, nasal, vaginal, skin, oral, bladder, placenta, breast, scalp, ear, eye, kidney, lungs, and nail tissues. Accordingly, the biological sample can also be a nasal swab sample, an oral mucosal swab sample, or a vaginal swab sample, without limitation.

Hypervariable regions of 16S rRNA genes can be sequences. In some embodiments, the entire 16S region in each genome in the sample is amplified. In some embodiments, no amplification is required, and the genomic sequences are directly sequenced. In some embodiments, each specific hypervariable region is amplified and/or sequenced, such as each of the V1-V9 hypervariable subregions. DNA amplification and sequencing can be carried out with methods known in the art, such as polymerase chain reactions (PCR), next generation sequencing (NGS) and deep sequencing, without limitation.

16S ribosomal RNA (or 16S rRNA) is the RNA component of the 30S subunit of a prokaryotic ribosome (SSU rRNA). It binds to the Shine-Dalgarno sequence and provides most of the SSU structure. The 16S rRNA gene can be used for phylogenetic studies as it is highly conserved between different species of bacteria and archaea. 16S rRNA gene can also be used as a reliable molecular clock because 16S rRNA sequences from distantly related bacterial lineages are shown to have similar functionalities.

In addition to highly conserved sequences, 16S rRNA genes also contain hypervariable regions that can provide species-specific signature sequences useful for identification of bacteria. The bacterial 16S gene contains nine hypervariable regions (V1-V9), ranging from about 30 to 100 base pairs long, that are involved in the secondary structure of the small ribosomal subunit. The degree of conservation varies widely between hypervariable regions, with more conserved regions correlating to higher-level taxonomy and less conserved regions to lower levels, such as genus and species.

In the event the 16S rRNA sequences are sequenced by fragments (e.g., by PCR), fragments can be trimmed and concatenated to form complete sequences. The concatenation may be assisted with whole gene sequences provided by databases, if needed.

In some embodiments, in addition to sequencing of the 16S rRNA hypervariable regions, the number of each hypervariable region in the 16S rRNA genes may be counted for each genome. The copy numbers can be readily obtained once the sequences are obtained. It is contemplated that the combination of 16S sequence along with the unique hypervariable region copy number achieved improved identification of microbial species/strains.

The 16S sequences and copy numbers can then be used to identify the microbial strain/species, by comparing such information to a suitable database, such as those described below. Type strains of 16S rRNA gene sequences for most bacteria and archaea are available on public databases, such as NCBI. However, the quality of the sequences found on these databases is often not validated. Therefore, secondary databases that collect only 16S rRNA sequences are widely used. The most frequently used databases include EzBioCloud, Ribosomal Database Project, SILVA and GreenGenes.

EzBioCloud database, formerly known as EzTaxon, consists of a complete hierarchical taxonomic system containing >60K bacteria and archaea species/phylotypes. Based on the phylogenetic relationship such as maximum-likelihood and OrthoANI, all species/subspecies are represented by at least one 16S rRNA gene sequence. The EzBioCloud database is systematically curated and updated regularly which also includes novel candidate species.

The Ribosomal Database Project (RDP) is a curated database that offers ribosome data along with related programs and services. The offerings include phylogenetically ordered alignments of ribosomal RNA (rRNA) sequences, derived phylogenetic trees, rRNA secondary structure diagrams and various software packages for handling, analyzing and displaying alignments and trees.

SILVA provides comprehensive, quality checked and regularly updated datasets of aligned small (16S/185, SSU) and large subunit (23S/285, LSU) ribosomal RNA (rRNA) sequences for all three domains of life as well as a suite of search, primer-design and alignment tools (Bacteria, Archaea and Eukarya).

GreenGenes is a quality controlled, comprehensive 16S rRNA gene reference database and taxonomy based on a de novo phylogeny that provides standard operational taxonomic unit sets.

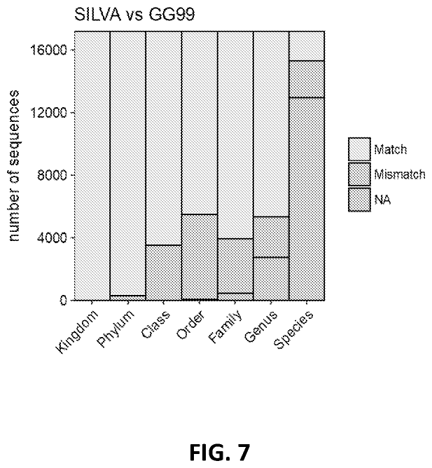

Based on the instant inventors' analysis, however, these databases disagreed in species assignment for more than half of identical, full-length 16S sequences (FIG. 7). Therefore, 16S sequence analysis using such databases is limited in mapping microbial sequences to the species level.

The instant disclosure provides a new database that includes further information and improved data structure for microbiome analysis. An example database, referred to as RExMapDB, is generated with the following example steps: (1) acquiring bacterial and/or archaeal genome sequences; (2) extracting 16S sequences from the sequences; (3) eliminating sequences that are not within hypervariable region of 16S, e.g., by using PCR primers to simulate amplification of the hypervariable region; (4) counting sequence variants and copy numbers of unique hypervariable region; (5) identifying a list of 16S rRNA gene sequences from bacterial and archaeal strains; and (6) cleaning the data, e.g., (i) if within a set of strains sharing the exact 16S hypervariable region, 99% or more strains share a single taxonomic rank (Family, Order, Class or Phylum), the remaining ≤1% outlier strains are removed; (ii) strains with double species annotations, such as “Psychrobacter immobilis Neisseria meningitidis” are removed.

In step (1), the bacterial and/or archaeal genome sequences can be readily acquired from generic sequence databases such as NCBI's GenBank, or 16S-specific ones such as EzBioCloud, Ribosomal Database Project, SILVA and GreenGenes. Depending on the database, step (2) may be optional, as step (3) may be carried out directly from the sequences. In some embodiments, the hypervariable regions can be extracted with suitable annotation; in some embodiments, the hypervariable regions can be identified with virtual PCR using PCR primers to simulate the amplification.

Step (4) here is unique to RExMapDB, which counts not only 16S hypervariable region sequence variants but also copy numbers of unique hypervariable regions. In some databases, such as the GenBank, such copy numbers may be derived from the whole genome sequences, but are not separately captured or recorded. Therefore, such databases cannot be used for the purpose of comparing the hypervariable region sequence and copy numbers.

In step (5), all identified 16S rRNA hypervariable regions, along with their copy numbers, are assembled to generate a list, which can go through additional clean-up in step (6). In an example clean-up step, outlier strains are removed. For instance, if within a set of strains sharing the exact 16S hypervariable region, 99% or more strains share a single taxonomic rank (Family, Order, Class or Phylum), then the remaining 1% outlier strains are removed. In another example, species or stains having multiple annotations can be considered to have misinformation and can be removed. Such clean-up can ensure higher quality sequence analysis and species/strain identification.

As provided, the database developed herein can be used to identify a microbial species or strain in a biological sample from a human subject. In some embodiments, before comparing the 16S data of a microbial species or strain to the database, the 16S data can be pre-processed. Example pre-processing steps include, without limitation, de-noising, un-trimming, merging and mapping.

De-noising, in an example, can be done using the partitioning algorithm such as those from DADA2⁵, to corrects sequenced amplicon errors. In an un-trimming process, consensus of suffixes of all sequences in the same partition is concatenated back with chimeric reads removed. In some embodiments, the abundance of the microbial genome is estimated based on the 16S sequences identified.

The pre-processed 16S hypervariable region sequences and copy numbers are then compared to the database to identify the corresponding microbial species or strains. An example process is as follows:
Pre-process 16S data of an individual (after optionally obtaining the sequences from a biological sample isolated from the individual in a lab)
   a. Pre-processing includes, for instance, de-noising, un-trimming, mapping to the database, and estimation of microbial abundance
   b. 16S sequencing reads from sequence files are merged, PCR primers removed, and the resulting sequences are quality filtered and trimmed to fixed length
   c. De-noising can be done using the partitioning algorithm from DADA2⁵, and then un-trimmed (i.e. consensus of suffixes of all sequences in the same DADA partition is concatenated back), with chimeric reads removed
The pre-processed unique sequences, are aligned to the database (e.g., RExMapDB), with retention of alignments with the highest alignment score.
An optimized constrained linear equation system was established using 16S sequence variation and copy number variation to infer strain identification and relative abundance from the observed read counts of the unique processed sequences.
The sequence matching against the database for a specific hypervariable region can be done using the BLAST algorithm. For each sequence, the best BLAST hits are saved, based on the alignment score (e.g., using parameters: match=5, mismatch=−4, gap open=−8, gap extend=−6), with a minimum percentage identity of 75% and word size of 50 nt.

This identification process can be carried out for each 16S rRNA gene identified in the biological sample. Accordingly, a listing of microbial species and/or strains can be identified for the biological sample. In some embodiments, each identified species or strain has an abundance or concentration in the biological sample.

As shown in the experimental examples, some core sets of gut microbes are shared by humans, and the core microbes are established soon after birth. Still further, it was observed that the core microbes closely correlate to BMI of subjects. The consistent presence of these core microbes across different demographic populations, throughout the lifespan of individuals, and their correlation to health conditions (current or future) make them uniquely suitable for identifying health risks.

A human subject may be identified as being deficient in a particular core microbe. For instance, the experimental data show that each human gut microbiome contained on average 13 of the core Operational Strain Units (OSUs), while 82% of individuals had at least 10 core OSUs and 97% of individuals had at least 5 core OSUs. The 15 OSUs identified in the examples represent 12 different species:
Eubacterium rectale,
Anaerostipes hadrus,
Blautia faecis M25,
Blautia obeum,
Blautia obeum/wexlerae,
Dorea longicatena,
Fusicatenibacter saccharivorans,
Roseburia inulinivorans,
Oscillibacter sp. ER4,
Romboutsia timonensis,
Faecalibacterium prausnitzii, including Operational Strain Units (OSU):
   Faecalibacterium prausnitzii (96%),
   Faecalibacterium prausnitzii (99%),
   Faecalibacterium prausnitzii [1], and
   Faecalibacterium prausnitzii [2], and
Gemmiger formicilis X2-56.

When a subject includes only 4 or even fewer of these OSU/species, it is likely that the subject is at least deficient in some of them. In some embodiments, the microbial species/strains identified from a subject, along with their abundance, are compared to a subject or a group of subjects having similar demographic background (e.g., race, gender, location of residence) so that a more precise comparison can be made. If this subject is missing (or having lower abundance for) a microbial species/strain as compared to the group, then the subject can be identified as be deficient in that species/strain.

In some embodiments, the subject is a minor, such as a subject that is 18, 17, 16, 15, 14, 13, 12, 11, 10, 9, 8, 7, 6, 5, 4, 3, 2 or 1 year(s) or younger. As shown in the experimental examples, the core species/strains are established shortly after birth, which are consistent throughout the lifespan. Therefore, the microbial species/strains identified in the minor can be projected to the adult life. Any deficiency observed can be used to guide treatment/dietary planning for the subject.

The instant disclosure, in some embodiments, also provides methods to treat a health condition associated with the deficiency identified, or guide the design of a dietary plan. For instance, if a subject is identified as being deficient in a particular microbial species or strain, medications or dietary supplements can be provided to the subject to remedy the deficiency.

Medications, foods and dietary supplements are available for such remedies. For instance, dairy products such as yogurt and kefir can improve the growth of gut microbiome. Another example is probiotics which are live microorganisms that are intended to have health benefits when consumed or applied to the body. They can be found in some fermented foods and dietary supplements. Probiotics can come in different forms and contain a variety of microorganisms.

Other foods and dietary items such as sauerkraut, apple cider vinegar, almonds, lentils, miso, fiber-rich foods, and salmon are also good candidates to improve the microbiome in a human subject.

### Kits and Packages, Software Programs

The methods described herein may be performed, for example, by utilizing pre-packaged diagnostic kits, such as those described below, comprising at least one probe or primer nucleic acid described herein, which may be conveniently used, e.g., to determine whether a subject has or is at risk of being deficient in certain microbial species.

In one embodiment, provided is a kit or package useful for identifying a patient as being likely or not likely to be deficient in microbial species, such as nucleic acid probes. In one embodiment, a kit further includes instructions for use. In one aspect, a kit includes a manual comprising reference gene expression levels.

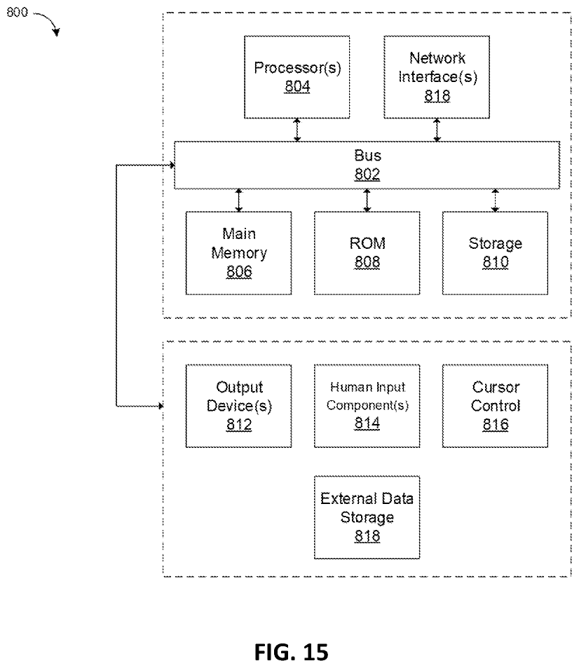

FIG. 15 is a block diagram that illustrates a computer system 800 upon which any embodiments of the present and related technologies may be implemented. The computer system 800 includes a bus 802 or other communication mechanism for communicating information, one or more hardware processors 804 coupled with bus 802 for processing information. Hardware processor(s) 804 may be, for example, one or more general purpose microprocessors.

The computer system 800 also includes a main memory 806, such as a random access memory (RAM), cache and/or other dynamic storage devices, coupled to bus 802 for storing information and instructions to be executed by processor 804. Main memory 806 also may be used for storing temporary variables or other intermediate information during execution of instructions to be executed by processor 804. Such instructions, when stored in storage media accessible to processor 804, render computer system 800 into a special-purpose machine that is customized to perform the operations specified in the instructions.

The computer system 800 further includes a read only memory (ROM) 808 or other static storage device coupled to bus 802 for storing static information and instructions for processor 804. A storage device 810, such as a magnetic disk, optical disk, or USB thumb drive (Flash drive), etc., is provided and coupled to bus 802 for storing information and instructions.

The computer system 800 may be coupled via bus 802 to a display 812, such as a LED or LCD display (or touch screen), for displaying information to a computer user. An input device 814, including alphanumeric and other keys, is coupled to bus 802 for communicating information and command selections to processor 804. Another type of user input device is cursor control 816, such as a mouse, a trackball, or cursor direction keys for communicating direction information and command selections to processor 804 and for controlling cursor movement on display 812. In some embodiments, the same direction information and command selections as cursor control may be implemented via receiving touches on a touch screen without a cursor. Additional data may be retrieved from the external data storage 818.

The computer system 800 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and maybe originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage.

The computer system 800 may implement the techniques described herein using customized hard-wired logic, one or more ASICs or FPGAs, firmware and/or program logic which in combination with the computer system causes or programs computer system 800 to be a special-purpose machine. According to one embodiment, the techniques herein are performed by computer system 800 in response to processor(s) 804 executing one or more sequences of one or more instructions contained in main memory 806. Such instructions may be read into main memory 806 from another storage medium, such as storage device 810. Execution of the sequences of instructions contained in main memory 806 causes processor(s) 804 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 810. Volatile media includes dynamic memory, such as main memory 806. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 802. Transmission media can also take the form of acoustic or light waves, such as those generated during radio-wave and infra-red data communications.

Various forms of media may be involved in carrying one or more sequences of one or more instructions to processor 804 for execution. For example, the instructions may initially be carried on a magnetic disk or solid-state drive of a remote computer. The remote computer can load the instructions into its dynamic memory and send the instructions over a telephone line using a component control. A component control local to computer system 800 can receive the data on the telephone line and use an infra-red transmitter to convert the data to an infra-red signal. An infra-red detector can receive the data carried in the infra-red signal and appropriate circuitry can place the data on bus 802. Bus 802 carries the data to main memory 806, from which processor 804 retrieves and executes the instructions. The instructions received by main memory 806 may retrieve and execute the instructions. The instructions received by main memory 806 may optionally be stored on storage device 810 either before or after execution by processor 804.

The computer system 800 also includes a communication interface 818 coupled to bus 802. Communication interface 818 provides a two-way data communication coupling to one or more network links that are connected to one or more local networks. For example, communication interface 818 may be an integrated services digital network (ISDN) card, cable component control, satellite component control, or a component control to provide a data communication connection to a corresponding type of telephone line. As another example, communication interface 818 may be a local area network (LAN) card to provide a data communication connection to a compatible LAN (or WAN component to communicated with a WAN). Wireless links may also be implemented. In any such implementation, communication interface 818 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world-wide packet data communication network now commonly referred to as the “Internet”. Local network and Internet both use electrical, electromagnetic or optical signals that carry digital data streams. The signals through the various networks and the signals on network link and through communication interface 818, which carry the digital data to and from computer system 800, are example forms of transmission media.

The computer system 800 can send messages and receive data, including program code, through the network(s), network link and communication interface 818. In the Internet example, a server might transmit a requested code for an application program through the Internet, the ISP, the local network and the communication interface 818.

The received code may be executed by processor 804 as it is received, and/or stored in storage device 810, or other non-volatile storage for later execution. Each of the processes, methods, and algorithms described in the preceding sections may be embodied in, and fully or partially automated by, code modules executed by one or more computer systems or computer processors comprising computer hardware. The processes and algorithms may be implemented partially or wholly in application-specific circuitry.

The various features and processes described above may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the embodiments should, therefore, be construed in accordance with the appended claims and any equivalents thereof.

The various operations of example methods described herein may be performed, at least partially, by one or more processors that are temporarily configured (e.g., by software) or permanently configured to perform the relevant operations. Similarly, the methods described herein may be at least partially processor-implemented, with a particular processor or processors being an example of hardware. For example, at least some of the operations of a method may be performed by one or more processors. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Mapping of Gut Microbial Species from 16S Data Across 29,000 Individuals from Around the World

The human gut microbiome has been linked to health and disease. Investigation of the human microbiome has largely employed 16S amplicon sequencing, with limited ability to distinguish microbes at the species level. Herein, we develop Reference-based Exact Mapping of microbial amplicon variants (RExMap), that enables mapping of microbial species from standard 16S sequencing data. RExMap analysis of 16S data captures ˜75% of microbial species identified by whole-genome shotgun sequencing, despite hundreds-fold less sequencing depth. RExMap re-analysis of existing 16S data from 29,349 individuals across sixteen regions around the world, reveals a detailed landscape of gut microbial species across populations and geography. Moreover, RExMap identifies a core set of fifteen gut microbes shared by humans. Core microbes are established soon after birth and closely associate with BMI across multiple independent studies. RExMap and The World Microbiome Database are presented as resources with which to explore the role of the human microbiome.

Herein we develop and benchmark a pipeline, Reference-based Exact Mapping of microbial amplicon variants (RExMap), for mapping microbial species from 16S data. RExMap establishes and makes use of a newly generated database (RExMapDB) that includes variants and copy numbers of 16S hypervariable regions for more than 170,000 bacterial and archaeal isolate strains currently within the NCBI Genome database. The aim of RExMap is to offer a complementary approach which bypasses traditional taxonomic assignments, and instead maps 16S sequences to exactly or near-exactly matched isolate strains, regardless of whether their precise taxonomy is presently known. RExMap analysis of 16S data is found to capture ˜70% of the microbial species identified by deep WGS of human fecal samples, at less than 1% of the sequencing depth.

RExMap further enables re-analysis of existing 16S data, with homogenization of sequencing data and large-scale meta-analyses. We leveraged the enormous volume of existing 16S data in public repositories to generate an aggregated database of all 16S sequencing data containing raw 16S sequences that we can re-analyze using RExMap. We obtained more than 10,000 previously published studies, spanning a record 1,000,000 16S microbial samples and consisting of 25 TB of compressed sequencing data. Application of RExMap to The World Microbiome Database for re-analysis of existing human gut microbiome 16S data across 29,349 individuals from ten studies covering sixteen regions of the world reveals a detailed landscape of the human gut microbiome, encompassing tens of thousands of unique microbes. We find gut microbial species segregate between the Westernized and non-Westernized regions and discover unique microbial species specific to certain geographic regions. Moreover, we discovered a set of core microbial species—confirmed orthogonally by shotgun whole genome sequencing—highly shared amongst humans, irrespective of individual region, lifestyle and environment, that are established soon after birth and likely contribute to nutritional metabolism.

RExMap and The World Microbiome Database are a rich resource with which to explore the role of the gut microbiome in human biology.

### Materials and Methods

### RExMapDB Construction

RExMapDB is generated by a series of Python scripts. First, bacterial and archaeal genome assembly summary tables are downloaded from the NCBI Genome database FTP server. For each unique strain name, only one assembly is selected by prioritizing (i) the assembly level (Complete Genome >Chromosome >Scaffold >Contig), (ii) RefSeq category (reference genome >representative genome >no annotation) and (iii) latest release date. FASTA sequences and GFF annotations are downloaded for each assembly (total size ˜50 GB). 16S sequences are extracted from the assembly FASTA files using annotations from the corresponding GFF files. Standard PCR primers for each hypervariable region of interest are aligned to each FASTA file, simulating a PCR experiment in silico and eliminating some of the possible contaminants (such as human sequences) in genome assemblies. For each strain assembly, sequence variants and copy numbers of unique hypervariable region are counted by aligning hypervariable region-specific PCR primers to the assembly FASTA file. Next, a Python script calls NCBI web eUtils to query RefSeq database for “16s ribosomal RNA[Title]NOT uncultured[Title] AND (bacteria[Filter] OR archaea[Filter]) AND (1000[SLEN]: 2000[SLEN]) AND refseq[filter]” and yields a list of 16S rRNA gene sequences from isolated bacterial and archaeal strains. Sequence variants and copy numbers of the hypervariable region are extracted from these 16S sequences and merged with the ones extracted from full genomes. Finally, two quality control procedures are implemented to clean the data: (i) if within a set of strains sharing the exact 16S hypervariable region, 99% or more strains share a single taxonomic rank (Family, Order, Class or Phylum), the remaining £ 1% outlier strains are removed; (ii) strains with double species annotations, such as “Psychrobacter immobilis Neisseria meningitidis” are removed. These excluded strains are saved in a separate table for each hypervariable region. The current version of RExMapDB has 148 and 257 strains excluded for the V3-V4 and V4 hypervariable regions, respectively.

### RExMap Workflow

RExMap workflow consists of pre-processing of 16S data, de-noising, un-trimming, mapping to RExMapDB, and estimation of microbial abundance. For pre-processing, 16S sequencing reads from raw Illumina FASTQ files are merged, PCR primers removed, and the resulting sequences are quality filtered and trimmed to fixed length. Pre-processed sequences were de-noised using the partitioning algorithm from DADA2⁵, and then un-trimmed (i.e. consensus of suffixes of all sequences in the same DADA partition is concatenated back), with chimeric reads removed. These unique processed sequences, also known as amplicon sequence variants (ASVs), were aligned to RExMapDB, with retention of alignments with the highest alignment score. An optimized constrained linear equation system was established using 16S sequence variation and copy number variation to infer strain identification and relative abundance from the observed read counts of the unique processed sequences.

### Matching Sequences Against RExMapDB

The denoised sequences are matched against RExMapDB for a specific hypervariable region, using MegaBLAST from the blastn command line tool (bundled with the RExMap R package). Users can easily provide their own or a third-party reference database at this step. For each sequence, the best BLAST hits are saved, based on the alignment score (using parameters: match=5, mismatch=−4, gap open=−8, gap extend=−6), with a minimum percentage identity of 75% and word size of 50 nt. RExMap provides pre-generated databases for commonly used V4 and V3-V4 hyper-variable regions. For exact (100%) matches to the database, copy number information of each hyper-variable region is retrieved from a separate table.

### Abundance Estimation of Operational Strain Units (OSUs)

The abundance estimation of OSUs is split into three separate parts. (1) Sequences with <100% matches to the database are each assigned a single OSU with raw count equal to the sequence raw count. Exact 100% matches to database strains are further processed in two scenarios. (2) An OSU contains strains with the exact same sequence variant, with potentially different copy numbers. For example, one OSU could contain a single sequence variant v, with 3 different strains having copy numbers 1, 3 and 5. In that case the copy number of that OSU is assigned an integer value of an average copy number over all strains, so the OSU spectrum is {v: 3} in this example. (3) An OSU contains multiple sequence variants, e.g., v₁ and v₂, with copy numbers 5 and 1: {v₁: 5, v₂: 1}. Since multiple strains (and OSUs) can share the exact same sequence variants, the abundances of each OSU are estimated using a constrained linear model, as follows. The abundance sᵢ of each sequence variant i can be obtained by summing up contributions aᵢⱼ from each OSU j: sᵢ=Σⱼ₌₁^{m}aᵢⱼ\tilde{x}ⱼ, where aᵢⱼ is the copy number of sequence variant i in the OSU j, m is the total number of OSUs in the database and \tilde{x}ⱼ is the actual (unknown) count of that OSU and i∈{1, . . . , n} with n being the total number of denoised sequences in a specific sample. This system of equations can be represented in matrix form as A\tilde{X}=S, e.g., for n=3 and m=2:

$${\underbrace{( \begin{matrix}{a}_{11} & {a}_{12} & {a}_{13} \\ {a}_{21} & {a}_{22} & {a}_{23}\end{matrix} )}}_{copy numbers} \cdot {\underbrace{( \begin{matrix}{\tilde{x}}_{1} \\ {\tilde{x}}_{2} \\ {\tilde{x}}_{3}\end{matrix} )}}_{OSU counts} = {\underbrace{( \begin{matrix}{s}_{1} \\ {s}_{2}\end{matrix} )}}_{\begin{matrix}sequence \\ counts\end{matrix}}$$

where each column of the A matrix contains the copy number of each OSU for each detected sequence (its spectrum). Due to the uncertainty in the sequence counts sᵢ, in all practical cases this system is overdetermined and does not have an exact solution. However, an estimate for the exact solution can be obtained by finding OSU counts X that minimize the square of the residuals: ƒ(X; A, S)=∥Ax−B∥².

The issue with this solution is that it is very prone to overfitting. For example, assume a sample contains the strain with two sequence variants with copy numbers 3 and 1, i.e., a spectrum {v₁: 3, v₂: 1}, and that experimentally we obtain sequence counts of 279 and 102 for these two variants, respectively. It is often the case that a database contains similar strains that have only variant v₁ or v₂. In this case the optimal solution would have count 279/3=93 for the strain with {v₁: 3, v₂: 1} spectrum and 102−93=9 for the strain with {v₂: 1} spectrum. This is an example of overfitting due to the uncertainty in counts. To address this, we added a regularization term to the minimization function:

$$min [ f ( X ; A , S ) ] = min [ {\sum }_{i = 1}^{n} {( {\sum }_{j = 1}^{m} {α}_{ij} {x}_{j} - {s}_{i} )}^{2} + {\sum }_{j = 1}^{m} {α}_{j}^{2} H ( {x}_{j} ) ] .$$

The first term in the brackets is the sum of all the squared residuals ∥Ax −B∥², while the second regularization term adds a cost αⱼ to the introduction of each new OSU j. H(x) is a Heaviside step function. We solve this minimization problem in two steps to speed up computation. First, the solution is obtained for the non-regularized function using a function lsei from an R package limSolve, with constraint xⱼ≥0 for all j. This part eliminates large number of OSUs from the database that are not present in a specific dataset being analyzed, and therefore a large number of columns from the A matrix. To speed up the minimization in the next step, we transform the reduced A matrix into a block-matrix form and perform minimization on each independent block separately. The block-matrix transformation is done by converting the rows and columns of matrix A into an undirected graph, using an R package igraph⁴⁹. In this graph, each row is connected to a column, if the copy number in that matrix field is greater than zero. Then, all the connected components of the graph are obtained using the function components and each of these components are used to extract block-matrices of A. Each block is optimized separately by setting the regularization weights αⱼ to the un-regularized solutions xⱼ. Then, the regularization term has the form Σⱼ₌₁^{m}xⱼ²H(xⱼ). The regularized solution is obtained using pso function from the R package pso (Particle Swarm Optimizer) with parameters maxit=10 and vectorize=TRUE. This function is re-initialized with a random seed and run 1000 times (can be changed using option pso_n). Out of the ensemble of solutions, the one with the lowest value of function ƒ is selected.

### Quality Control of Gut Microbiome Datasets

Samples and 16S amplicon reads from the ten studies were filtered out at various stages in the processing pipeline. Any sample containing less than 5,000 reads was discarded from further analysis. For 16S sequencing data, paired-end reads were discarded if they: (i) cannot not be merged using an overlap alignment with the two thresholds—minimum percent similarity of 75% and minimum overlap length of 50 nucleotides, (ii) contain 2 or more expected errors, (iii) match PhiX genome, (iv) contain any Ns, or (v) have any quality score 2 or less. After denoising, sequences were kept if they were present in 5 or more samples or had a median raw count greater than 100 in the samples in which they were present. OSUs with a mean abundance greater than 0.0001% in the 29,349 gut microbiomes were kept for analysis. All samples were de-bloomed following a previously established protocol¹⁸.

### Comparison of Microbial Species Mapped by MetaPhlan WGS Vs RExMap 16S Data

For Jovel et al¹⁵ and DIABIMMUNE³ datasets, MetaPhlAn2 was used by the authors to estimate species abundances, with default parameters. Species output by MetaPhlAn2 were matched with RExMap OSUs using exact names, with one exception: Ruminococcus obeum from MetaPhlAn2 was renamed to Blautia obeum. Since MetaPhlAn2 outputs single species names, each of these species names was matched as follows: typically, RExMap OSUs contain a single or a few species and thus the matching is one-to-one. In some cases, however, multiple OSUs may contain the same species. In these cases, all best matching OSUs containing that species name are pooled together into a larger OSU, with the abundances equal to the sum of all abundances of the pooled OSUs. The abundances of the OSUs matched to MetaPhlAn2 outputs were normalized to unity for comparison.

### Principal Coordinate Analysis (PCoA)

The counts from the OSU estimation step are normalized for each sample to 1 by dividing them by the total OSU count in each sample. These normalized counts are referred to as “abundances” or “relative abundances” (or % abundances if multiplied by 100). OSUs detected in 10 or fewer samples were filtered out. Abundances of undetected OSUs were imputed with the value of 10^{-6.23}, which ensured that the total abundance of all imputed OSUs did not exceed 1% of abundance of any sample. The distance metric between OSUs used was defined as (1−r)/2, where r is the Spearman correlation between log₁₀-transformed imputed abundances of each pair of OSUs. This resulted in a 15,877×15,877 distance matrix. To significantly speed up the computation of the matrix of this size, multi-dimensional scaling was performed using Microsoft Open R 3.5.3, which implements multithreaded eigen-analysis using R's base eigen( ) function. Lingoes correction was used to correct for negative eigenvalues. Finally, PCoA coordinates for each sample were calculated by transforming OSUs to the first three principal coordinates using the (15,877×3) transformation matrix obtained from the multi-dimensional scaling.

### Core OSU WGS Analysis

The prevalence and abundance of the core OSUs mapped by RExMap from 16S data of the 1,077 samples from TwinsUK were validated in a non-matching set of 209 samples from TwinsUK with WGS data²⁹. WGS data was analyzed by Kraken2/Bracken pipeline⁵⁰ using the quality control protocol applied in the American Gut Project¹⁸. The 100 nt paired-end reads were previously quality-filtered and had human sequences removed if they aligned to hg19 human genome²⁹. These processed reads were then assigned taxonomy using Kraken2. Kraken2 was run with the extended version of Kraken2 standard database, which also includes bacterial and archaeal genomes labelled as “Contig” or “Scaffold” level assemblies by the NCBI Genome database.

### BMI Association Analysis

The analysis was done on 3 large adult populations with available BMI, age and gender phenotypes, between 18 and 80 years of age and with BMI between 15 and 40 kg/m²: American Gut Project (N=9,621), Guangdong Gut Microbiome Project (N=6,748) and Twins UK 2014 (N=1,013). BMI categories were defined as: (i) underweight: BMI £ 18.5, (ii) normal: 18.5<BMI £ 25, (iii) overweight: 25<BMI £ 30, and (iv) obese: BMI >30. P-values were obtained from two-tailed Wilcox rank-sum tests. Linear regressions were performed of the form BMI˜log₁₀(xᵢ)+age+gender, where xᵢ is the relative abundance of the OSU i. Meta-analysis of effect size of individual core OSU on BMI is estimated with metagen function in the R meta package.

### Pooling Multiple Datasets

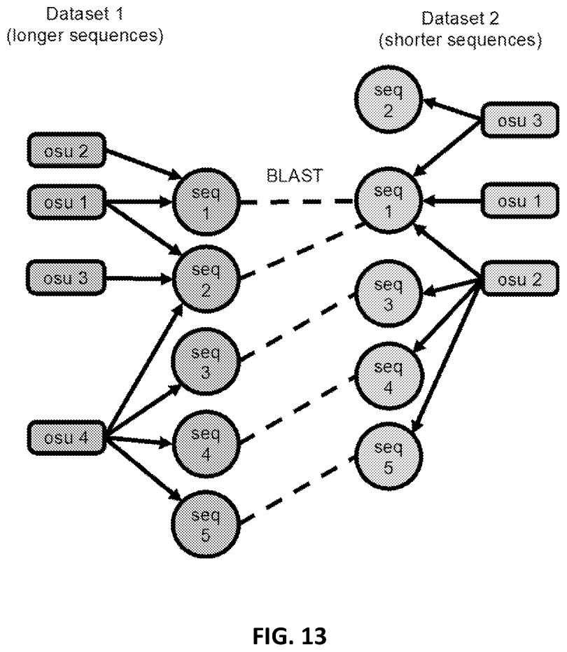

Two or more experiments can be pooled using a pool_rexmap_results function. The pooling algorithm takes as input final outputs of RExMap runs on two datasets, consisting of an abundance table (the output of the function abundance) and sequence table (output of the function osu_sequences) for each dataset. Multiple datasets are pooled in pairs, using Reduce function from base R: the first dataset pair is pooled, then the third dataset is pooled to the first two, the fourth one is pooled to the first three and so on. The problem schematic is shown in FIG. 13.

(OSUs and sequences corresponding to two datasets are shown in different colors). Pooling two datasets starts by using MegaBLAST to obtain a semi-global alignment (where gaps appearing as prefixes or suffixes are ignored) between all sequences from the two datasets that have 100% identity in the aligned region. MegaBLAST alignment is run with parameters match=1, mismatch=−2, gapopen=1, gapextend=1, perc_identity=100, max_target_seqs=1000, word_size=max(min_seq_len*0.5), where min_seq_len is the length of the shortest sequence in the dataset. Large word size and 100% similarity parameters ensure the alignment runs very fast (few minutes on a modem laptop) even when pooling datasets with very large number of sequences (100,000+).

The local alignments (where only shorter substrings of query and subject are matched) are filtered out from the BLAST output table (“outfmt 6”), by keeping only alignments that contain either (i) full query sequence (so query is a substring of subject), (ii) full subject sequence (so that subject is a substring of query), (iii) start of query sequence and an end of subject sequence (so that the beginning of subject and an end of query are the only gaps) or (iv) start of subject sequence and an end of query sequence (so that the beginning of query and an end of subject are the only gaps).

For each alignment in this table, a final sequence is chosen as one of the two that contains less information (is mapped to more unique strains), which is typically, but not always, a shorter sequence.

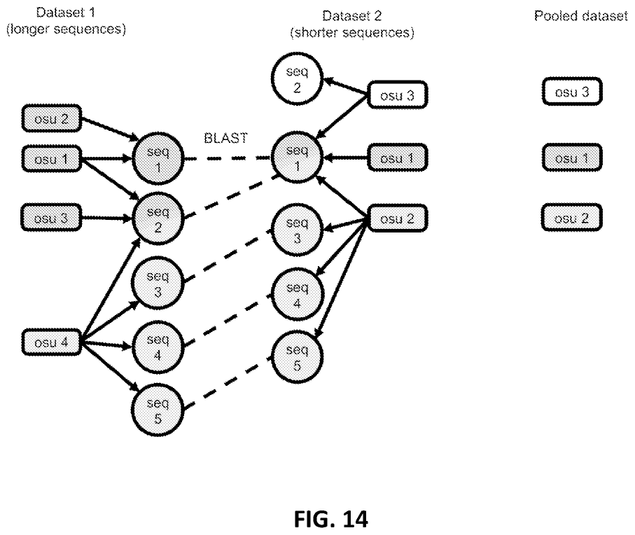

The alignment table is joined with tables cross-referencing the joined sequences with OSUs from each of the two datasets. OSUs are declared “identical” only when all sequences from OSU 1 from dataset 1 match to all sequences from OSU 1 from dataset 2. This allows pooling of samples with large differences in sequencing length, i.e., V3-V4 regions (˜420 nt) vs V4 regions (˜250 nt) and pooling of OSUs that have low identity to any of the reference sequences, because it ensures that they share an exact sequence match (up to prefix and suffix gaps). OSUs that are partially matched between datasets are kept separate, e.g., if only one of the two sequences from OSU 3 (dataset 2) is matched to OSU 2 (dataset 1), however the pooled matched sequence is taken as the shorter of two lengths. These cases are illustrated in FIG. 14.
and ensure that all the pooled output sequences are not identical up to prefix and suffix gaps, so can be pooled with further datasets.

### Results

### RExMap Workflow

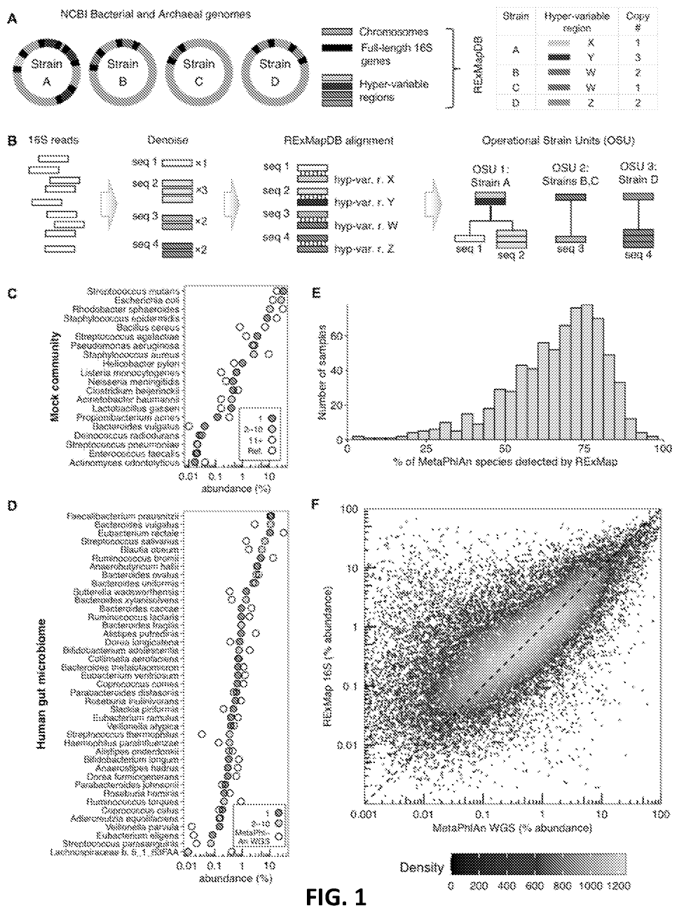

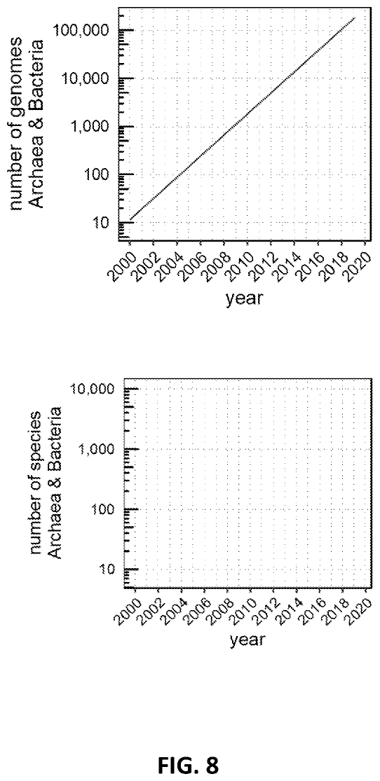

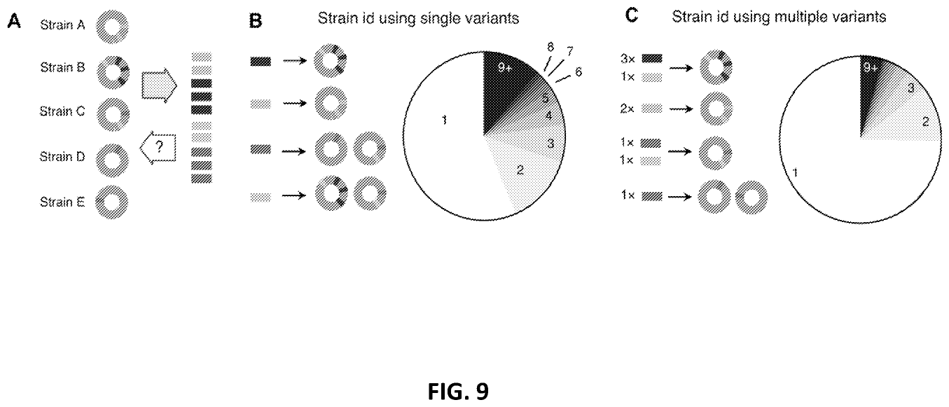

Ongoing work from the Human Microbiome Project (HMP)¹¹, other consortiums, and individual research groups have exponentially expanded the number of isolated and fully sequenced microbial genomes over the last decade, now totaling over 170,000 specific strains and 18,000 species (FIG. 8), with particular enrichment for microbes originating from the human gut. From these microbial genomes, we extracted the hypervariable region of 16S rRNA genes corresponding to the amplicon sequences obtained using universal PCR primers. Analysis of V3-V4 regions from all completely assembled microbial genomes revealed that ˜50% of sequences map to only a single microbial strain (FIG. 9). In addition, ˜30% of microbial strains we found exhibit multiple unique V3-V4 hypervariable regions and copy number variations, potentially enabling more precise identification of microbes through inclusion of this variant information. Leveraging of both sequence and copy number variation of the V3-V4 region improved microbial mapping, with 75% of sequences mapping to a single strain, and 94% of sequences corresponding to five or fewer strains (FIG. 9). As such, V3-V4 and V4 hypervariable regions for all 174,135 isolate microbial strains were curated in a BLAST Reference-based Exact Mapping DataBase (RExMapDB) (FIG. 1A). This reproducible, locally customizable, updateable and open-source database allows sequence mapping to known microbial strain isolates. To match experimentally derived 16S amplicon sequencing data to the RExMapDB, a modular RExMap workflow was established, consisting of pre-processing, denoising (see Methods), mapping to RExMapDB and estimation of microbial abundances.

Indistinguishable strains whose genomes share exact 16S hypervariable region sequence and copy number variants in RExMapDB were grouped together and termed an “Operational Strain Unit” (OSU) (FIG. 1B).

### RExMap Based Species Mapping

To determine the utility of RExMap for mapping microbial species from 16S sequencing data, we first benchmarked the approach using the Human Microbiome Project (HMP) 20-species staggered mock community¹⁴. Among the 20 species in the mock community, RExMap correctly identified all 20, with 13 OSUs comprised of a single microbial species and the remaining 7 OSUs composed of 2-10 species (median 5 species), with close estimation of relative abundance for all species (FIG. 1C). For the 13 OSUs mapped to a single species, RExMap was further able to identify 3 of the exact strains present in the mock community.

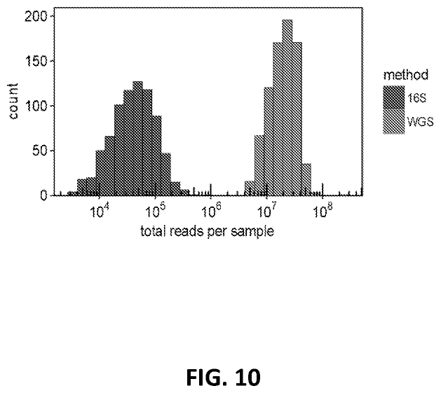

To evaluate the performance of RExMap for mapping microbial species from 16S amplicon data within a complex microbial community, we first examined a single human fecal sample previously subjected to paired 16S and WGS microbial sequencing¹⁵. From 16S V3-V4 sequencing, RExMap correctly identified all 41 known species found by WGS using MetaPhlAn¹⁶ with close estimation of relative abundance (FIG. 1D). Among the 41 OSUs, 26 contained only a single species (FIG. 1D). We next evaluated a dataset of 780 fecal samples with paired 16S and WGS data from the DIABIMMUNE study³. RExMap analysis of 16S data captured on average 70% species found in WGS by MetaPhlAn (FIG. 1E), despite less than 0.2% of the relative sequencing depth (FIG. 10). RExMap estimation of species abundance was further found to closely follow WGS measures across four orders of magnitude (FIG. 1F). Collectively, these data highlight the robust nature of RExMap for mapping of microbial species and estimation of abundance from 16S sequencing data.

### The World Microbiome Database

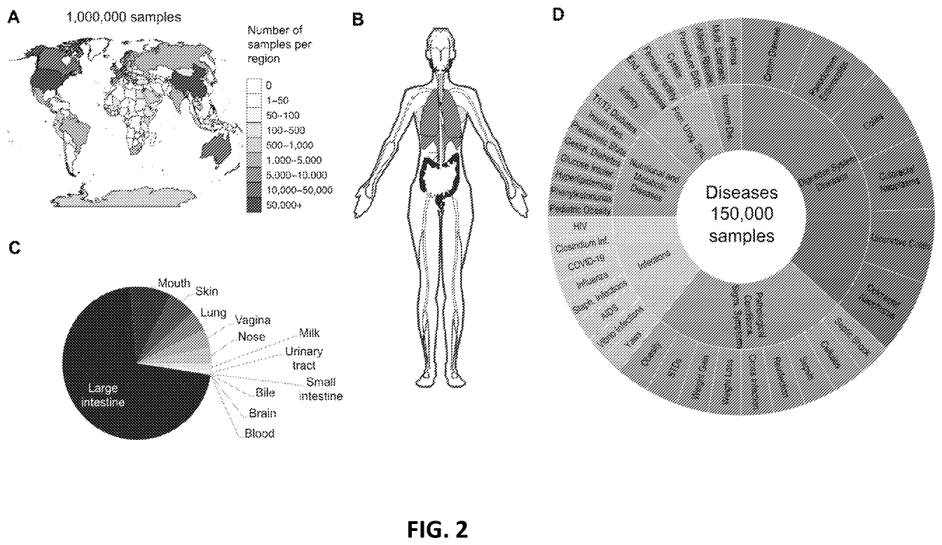

The World Microbiome Database is the most extensive and diverse microbial database aggregated to date. It consists of about 10,000 previously published studies indexed by Google Scholar, spanning a record 1,000,000 16S microbial samples and consisting of 25 TB compressed raw sequencing data. This continuously growing database contains samples reaching across all continents on Earth including Antarctica (FIG. 2A), sampling sites from human body, animals, plants, soil, ocean and air including dust samples from the International Space Station (FIG. 2B) and contains thousands of samples from extraordinary diverse range of diseases and conditions such as preterm birth, IBD, colorectal cancer, malnutrition, asthma, Crohn's disease, HIV, obesity, periodontitis, diabetes, liver diseases, sepsis, autoimmune diseases, arthritis, bacterial vaginosis as well as a spectrum of viral diseases including COVID-19 (FIG. 2C).

### RExMap Meta-Analysis of 29,349 Human Gut Microbiomes

To date, 16S amplicon sequencing has been performed across thousands of human fecal samples, with much of this data stored in publicly accessible centralized data repositories such as Qiita¹⁷, NCBI and EBI. Few studies, however, have attempted to aggregate multiple independent large-scale gut microbiome datasets to enable discovery of shared and divergent gut microbes across diverse human populations. To allow for cross-comparison and meta-analysis of human gut microbiome datasets across independent studies, RExMap was specifically designed to computationally scale for processing of 16S data from tens of thousands of samples. In addition, a pooling strategy was further developed to allow for matching OSUs from multiple datasets by aligning and harmonizing unique OSU sequences.

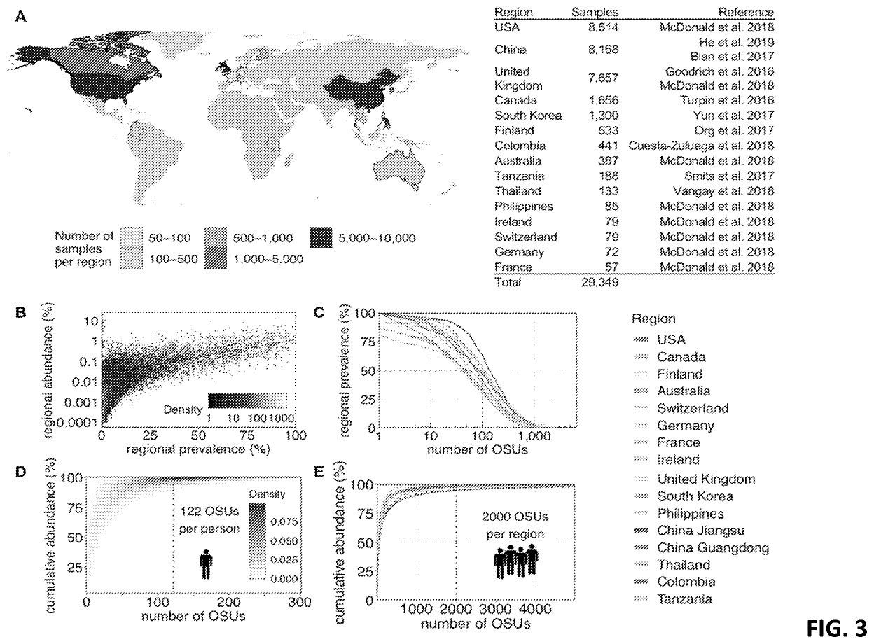

We applied RExMap for analysis of existing raw 16S sequencing data from ten population-scale human gut microbiome studies^{10,18-26} from The World Microbiome Database, collectively representing 29,349 adults with diverse lifestyle, dietary and environmental exposures, across sixteen distinct regions in fifteen countries: the United States, Canada, United Kingdom, Ireland, Finland, Switzerland, Germany, France, Australia, Colombia, China, South Korea, Thailand, Philippines, and hunter-gatherer tribes in Tanzania (FIG. 3A, Methods). From among these 29,349 individuals, a total of 17,786 unique OSUs were identified, with 12,078 (68%) OSUs containing a single microbial species and 97% of OSUs containing 5 or fewer microbial species. Within the full set of 17,786 OSUs, we identified a strong association between regional prevalence (fraction of samples in a region where an OSU is present) and regional mean abundance, with highly prevalent OSUs found to be highly abundant (FIG. 3B). Within each region across the world, only ˜100 OSUs were found to be more than 50% prevalent (FIG. 3C). Moreover, despite the large number of OSUs captured across the 29,349 individuals, 99% of cumulative microbial abundance within a single individual was found to be comprised on average of only 122 different OSUs (FIG. 3D). Within any of the sixteen geographic regions, ˜2,000 OSUs accounted for greater than 95% of the gut microbiome by relative abundance (FIG. 3E). This suggests that despite the influence of diverse lifestyles, geography, diets and environmental exposures, the overall number of RExMap-identified gut microbes present in individuals and populations was remarkably consistent across geographic regions.

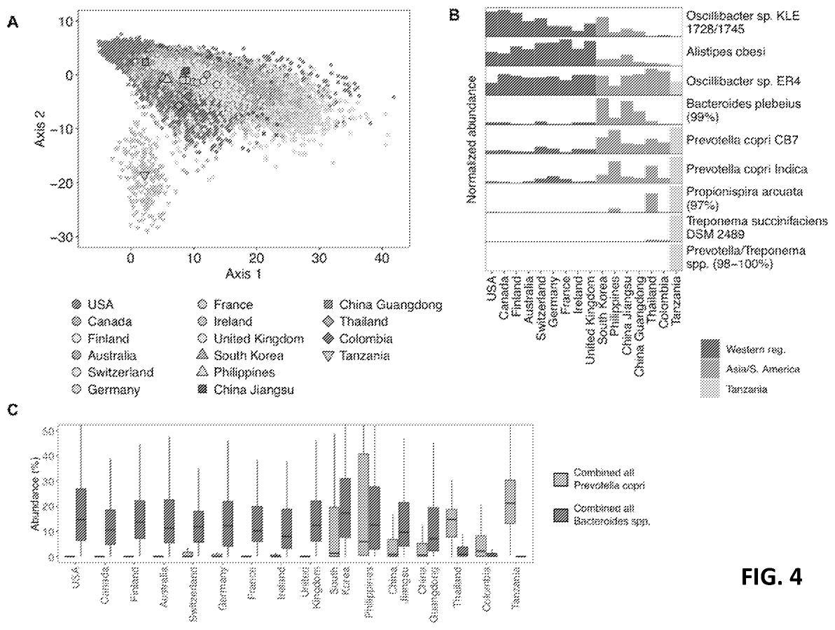

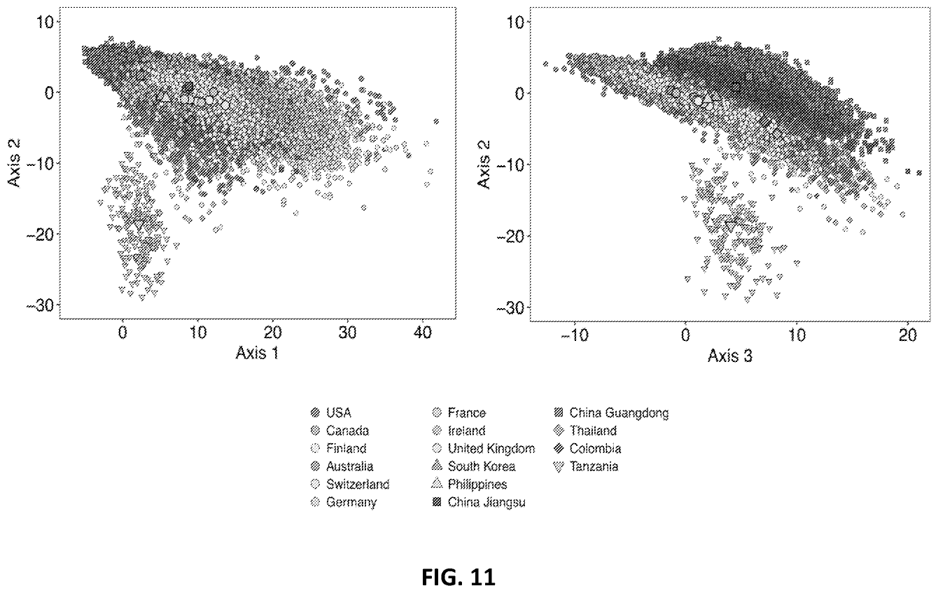

Principal coordinate analysis (PCoA) of all 29,349 gut microbiomes revealed that hunter-gatherers from Tanzania were distinct in their microbiomes relative to other regions of the world (FIG. 4A). Further separation was observed among Westernized countries (USA, Canada, UK, Ireland, Finland, Switzerland, Germany, France, Australia) and Asian/South American countries (South Korea, Philippines, Thailand, China, and Colombia) along the second and third principal coordinates (FIG. 11), potentially due to shared ethnic, lifestyle, and environmental factors among countries within each group. We further investigated the top OSUs contributing to regional variation along each of the first three principal coordinates (FIG. 4B). Among these OSUs, Oscillibacter sp. KLE1728/1745 and Alistipes obesi were abundant in Westernized countries, while Bacteroides plebeius, a microbe previously shown to metabolize dietary seaweed²⁷, was mostly abundant in Asian countries. Propionispira arcuata was only shared among gut microbiomes from Philippines, Thailand, and Tanzania, whereas unique Treponema and Prevotella species were only found in gut microbiomes from the hunter-gatherers in Tanzania (FIG. 4B). Two OSUs present in all cohorts, Prevotella copri CB7 and Prevotella copri indica were found to be the leading contributors to the separation between Westernized and non-Westernized countries (FIG. 4B). Previous studies have reported that gut microbes within the Prevotella genus may substitute for Bacteroides, both of which belong to the order Bacteroidales²⁸. Total abundance for these two Prevotella copri strains was indeed balanced with all Bacteroides across the sixteen regions, consistent with functional substitution.

Prevotella copri was more abundant in non-Westernized countries, with an abundance as high as 20% in hunter-gatherers from Tanzania, in contrast to <1% mean abundance in all the Westernized countries (FIG. 4C). These data suggest that across the world specific gut microbial species may exhibit regional variation.

### Core Human Gut Microbes

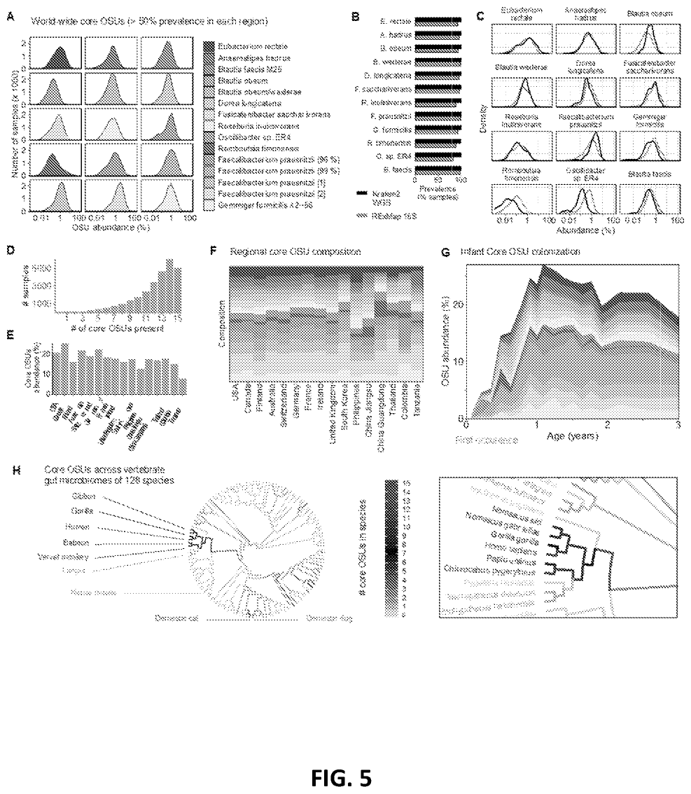

While differences in diet, lifestyle, and environment may influence regional variation in the gut microbiome¹⁹, it remains unknown whether a specific core set of gut microbes are conserved among all humans. From among the 29,349 human gut microbiomes examined, 15 OSUs mapping to 12 species, were found to be present in greater than 50% of the population within each of the sixteen geographic regions (FIG. 5A), and were therefore termed “core OSUs”. Prevalence and abundance of all 12 core species were further confirmed using WGS data analyzed by Kraken2 in a subset of TwinsUK samples²⁹ (FIG. 5B-C).

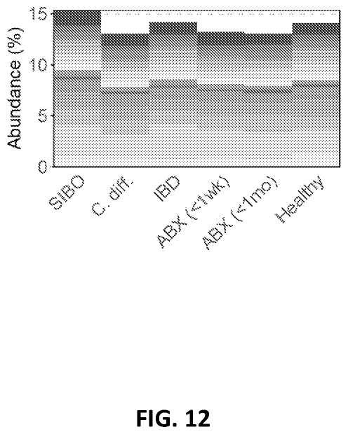

Each human gut microbiome was found to contain on average 13 of the core OSUs, while 82% of individuals had at least 10 core OSUs and 97% of individuals had at least 5 core OSUs (FIG. 5D). Core OSUs collectively accounted for 8-26% of the total gut microbiome in each region by relative abundance (FIG. 5E). Notably, all core OSUs were found to be Firmicutes belonging to the order Clostridiales, suggesting that specific bacterial species within other Phyla may be interchanged and substituted for one another across regions. Interestingly, core OSUs exhibited remarkable stability in regional-average composition across all regions (FIG. 5F), with ratio-metric values maintained even in individuals with dysbiosis including small intestinal bacterial overgrowth, C. difficile infection, inflammatory bowel disease, and recent antibiotic usage (FIG. 12). To determine at what age the core gut OSUs are established in humans, we examined a dataset of 222 infants with serial sampling throughout early life³. Each of the 15 core OSUs was found to be present in at least one infant within 3 months, and all 15 core OSUs reached a stable relative composition comparable to adults within the first year of life (FIG. 5G).

Given that the core OSUs appear highly conserved among humans, we next asked whether these microbes are shared among other vertebrate species. Across 128 diverse vertebrate species³⁰, core OSUs were found to be highly conserved in non-human primates, including langurs, gibbons, gorillas, baboons and monkeys (FIG. 5H), but were virtually absent outside of primates. Sporadic core OSUs were observed in vertebrates traditionally found in close proximity to humans, including domestic cat and domestic dog (FIG. 5H).

The conservation of the core OSUs among diverse populations and their appearance in developing infants in close temporal relation to diversification of oral feeding suggest a potential role for core OSUs in nutrient utilization and metabolic homeostasis.

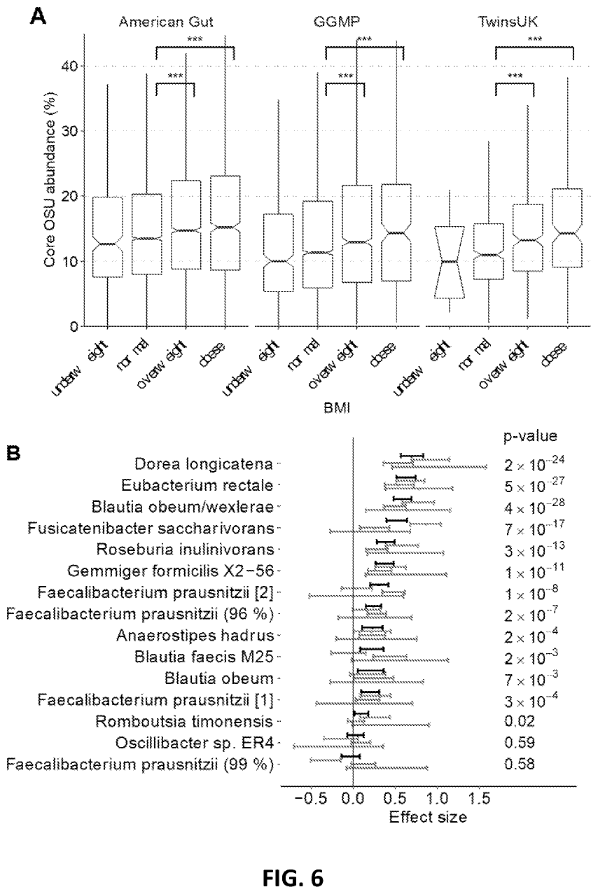

We therefore investigated the association between core OSUs and body mass index (BMI) in three diverse studies—the American Gut Project¹⁸, Guangdong Gut Microbiome Project¹⁹, and TwinsUK³¹, totaling 17,382 individuals. The cumulative abundance of all 15 core OSUs was found to increase across the entire weight spectrum, from underweight (BMI <18.5) and normal weight (BMI 18.5-25), to overweight (BMI 25-30) and obese (BMI >30) individuals and was consistent across all three diverse populations (FIG. 6A). Among individual core OSUs, 11 of 15 were found to increase in relation to BMI across all three populations (FIG. 6B). Collectively these data identify a set of shared gut microbes present across all human populations that are closely linked to body mass.

### REFERENCES

1. Turnbaugh, P. J. et al. A core gut microbiome in obese and lean twins. Nature 457, 480-484 (2009).
2. Matson, V. et al. The commensal microbiome is associated with anti-PD-1 efficacy in metastatic melanoma patients. Science 359, 104-108 (2018).
3. Vatanen, T. et al. Variation in Microbiome LPS Immunogenicity Contributes to Autoimmunity in Humans. Cell 165, 842-853 (2016).
4. Bolyen, E. et al. Reproducible, interactive, scalable and extensible microbiome data science using QIIME 2. Nat. Biotechnol. 1 (2019) doi:10.1038/s41587-019-0209-9.
5. Callahan, B. J. et al. DADA2: High-resolution sample inference from Illumina amplicon data. Nat. Methods 13, 581-583 (2016).
6. Schloss, P. D. et al. Introducing mothur: Open-Source, Platform-Independent, Community-Supported Software for Describing and Comparing Microbial Communities. Appl. Environ. Microbiol. 75, 7537-7541 (2009).
7. DeSantis, T. Z. et al. Greengenes, a Chimera-Checked 16S rRNA Gene Database and Workbench Compatible with ARB. Appl. Environ. Microbiol. 72, 5069-5072 (2006).
8. Quast, C. et al. The SILVA ribosomal RNA gene database project: improved data processing and web-based tools. Nucleic Acids Res. 41, D590-D596 (2012).
9. Cole, J. R. et al. Ribosomal Database Project: data and tools for high throughput rRNA analysis. Nucleic Acids Res. 42, D633-D642 (2014).
10. Goodrich, J. K. et al. Genetic Determinants of the Gut Microbiome in UK Twins. Cell Host Microbe 19, 731-743 (2016).
11. The Human Microbiome Project Consortium et al. Structure, function and diversity of the healthy human microbiome. Nature 486, 207-214 (2012).
12. Callahan, B. J. et al. High-throughput amplicon sequencing of the full-length 16S rRNA gene with single-nucleotide resolution. (2018) doi:10.1101/392332.
13. Johnson, J. S. et al. Evaluation of 16S rRNA gene sequencing for species and strain-level microbiome analysis. Nat. Commun. 10, 5029 (2019).
14. Zheng, W. et al. An accurate and efficient experimental approach for characterization of the complex oral microbiota. Microbiome 3, (2015).
15. Jovel, J. et al. Characterization of the Gut Microbiome Using 16S or Shotgun Metagenomics. Front. Microbiol. 7, (2016).
16. Segata, N. et al. Metagenomic microbial community profiling using unique clade-specific marker genes. Nat. Methods 9, 811-814 (2012).
17. Gonzalez, A. et al. Qiita: rapid, web-enabled microbiome meta-analysis. Nat. Methods 15, 796-798 (2018).
18. McDonald, D. et al. American Gut: an Open Platform for Citizen-Science Microbiome Research. mSystems (2018) doi:10.1101/277970.
19. He, Y. et al. Regional variation limits applications of healthy gut microbiome reference ranges and disease models. Nat. Med. 24, 1532 (2018).
20. Bian, G. et al. The Gut Microbiota of Healthy Aged Chinese Is Similar to That of the Healthy Young. mSphere 2, e00327-17 (2017).
21. Turpin, W. et al. Association of host genome with intestinal microbial composition in a large healthy cohort. Nat. Genet. 48, 1413-1417 (2016).
22. Yun, Y. et al. Comparative analysis of gut microbiota associated with body mass index in a large Korean cohort. BMC Microbiol. 17, (2017).
23. Org, E. et al. Relationships between gut microbiota, plasma metabolites, and metabolic syndrome traits in the METSIM cohort. Genome Biol. 18, 70 (2017).
24. Cuesta-Zuluaga, J. de la et al. Gut microbiota is associated with obesity and cardiometabolic disease in a population in the midst of Westernization. Sci. Rep. 8, 11356 (2018).
25. Smits, S. A. et al. Seasonal cycling in the gut microbiome of the Hadza hunter-gatherers of Tanzania. Science 357, 802-806 (2017).
26. Vangay, P. et al. US Immigration Westernizes the Human Gut Microbiome. Cell 175, 962-972.e10 (2018).
27. Hehemann, J.-H. et al. Transfer of carbohydrate-active enzymes from marine bacteria to Japanese gut microbiota. Nature 464, 908-912 (2010).
28. Yatsunenko, T. et al. Human gut microbiome viewed across age and geography. Nature 486, 222-227 (2012).
29. Xie, H. et al. Shotgun Metagenomics of 250 Adult Twins Reveals Genetic and Environmental Impacts on the Gut Microbiome. Cell Syst. 3, 572-584.e3 (2016).
30. Youngblut, N. D. et al. Host diet and evolutionary history explain different aspects of gut microbiome diversity among vertebrate clades. Nat. Commun. 10, 1-15 (2019).
31. Goodrich, J. K. et al. Human Genetics Shape the Gut Microbiome. Cell 159, 789-799 (2014).
32. Thompson, L. R. et al. A communal catalogue reveals Earth's multiscale microbial diversity. Nature (2017) doi:10.1038/nature24621.
33. Cani, P. D. Human gut microbiome: hopes, threats and promises. Gut 67, 1716-1725 (2018).
34. Blin, K. et al. antiSMASH 5.0: updates to the secondary metabolite genome mining pipeline. Nucleic Acids Res. 47, W81-W87 (2019).
35. Pasolli, E. et al. Extensive Unexplored Human Microbiome Diversity Revealed by Over 150,000 Genomes from Metagenomes Spanning Age, Geography, and Lifestyle. Cell (2019) doi:10.1016/j.cell.2019.01.001.
36. Machiels, K. et al. A decrease of the butyrate-producing species Roseburia hominis and Faecalibacterium prausnitzii defines dysbiosis in patients with ulcerative colitis. Gut 63, 1275-1283 (2014).
37. Sokol, H. et al. Faecalibacterium prausnitzii is an anti-inflammatory commensal bacterium identified by gut microbiota analysis of Crohn disease patients. Proc. Natl. Acad. Sci. 105, 16731-16736 (2008).
38. Lopez-Siles, M., Duncan, S. H., Garcia-Gil, L. J. & Martinez-Medina, M. Faecalibacterium prausnitzii: from microbiology to diagnostics and prognostics. ISME J. 11, 841-852 (2017).
39. Group, T. N. H. W. et al. The NIH Human Microbiome Project. Genome Res. 19, 2317-2323 (2009).
40. Takada, T., Kurakawa, T., Tsuji, H. & Nomoto, K. Fusicatenibacter saccharivorans gen. nov., sp. nov., isolated from human faeces. Int. J. Syst. Evol. Microbiol. 63, 3691-3696 (2013).
41. Ricaboni, D., Mailhe, M., Khelaifia, S., Raoult, D. & Million, M. Romboutsia timonensis, a new species isolated from human gut. New Microbes New Infect. 12, 6-7 (2016).
42. Rettedal, E. A., Gumpert, H. & Sommer, M. O. A. Cultivation-based multiplex phenotyping of human gut microbiota allows targeted recovery of previously uncultured bacteria. Nat. Commun. 5, 1-9 (2014).
43. Park, S.-K., Kim, M.-S. & Bae, J.-W. Blautia faecis sp. nov., isolated from human faeces. Int. J. Syst. Evol. Microbiol. 63, 599-603 (2013).
44. Mahowald, M. A. et al. Characterizing a model human gut microbiota composed of members of its two dominant bacterial phyla. Proc. Natl. Acad. Sci. U.S.A 106, 5859-5864 (2009).
45. Kasai, C. et al. Comparison of the gut microbiota composition between obese and non-obese individuals in a Japanese population, as analyzed by terminal restriction fragment length polymorphism and next-generation sequencing. BMC Gastroenterol. 15, 100 (2015).
46. Brahe, L. K. et al. Specific gut microbiota features and metabolic markers in postmenopausal women with obesity. Nutr. Diabetes 5, e159-e159 (2015).
47. Ottosson, F. et al. Connection Between BMI-Related Plasma Metabolite Profile and Gut Microbiota. J. Clin. Endocrinol. Metab. 103, 1491-1501 (2018).
48. Gomes, A. C., Hoffmann, C. & Mota, J. F. The human gut microbiota: Metabolism and perspective in obesity. Gut Microbes 9, 308-325 (2018).
49. Csardi, G. & Nepusz, T. The igraph software package for complex network research. 9.
50. Wood, D. E., Lu, J. & Langmead, B. Improved metagenomic analysis with Kraken 2. Genome Biol. 20, 257 (2019).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for improving the health of a human subject, comprising:
obtaining a biological sample from the human subject, wherein the biological sample comprises microbial species residing in the human subject;
sequencing hypervariable regions of 16S rRNA genes in bacterial genomes in the biological sample, and counting the copy number of each hypervariable region in the 16S rRNA genes in each genome;
comparing the hypervariable region sequences and copy numbers thereof in the 16S rRNA genes to a database comprising hypervariable region sequences and copy numbers thereof in the 16S rRNA genes of different bacterial species, to identify the bacterial species of the 16S rRNA genes;
comparing the identified bacterial species to common bacterial species found in human subjects, to identify bacterial species deficient in the human subject; and
administering to the human subject a dietary supplement supplying, or promoting the growth of, the deficient bacterial species.

2. The method of claim 1, wherein the common bacterial species comprise at least 5 selected from the group consisting of:
Eubacterium rectale,
Anaerostipes hadrus,
Blautia faecis,
Blautia obeum,
Blautia obeum/wexlerae,
Dorea longicatena,
Fusicatenibacter saccharivorans,
Roseburia inulinivorans,
Oscillibacter sp.,
Romboutsia timonensis,
Faecalibacterium prausnitzii, and
Gemmiger formicilis.

3. The method of claim 2, wherein the common bacterial species comprise at least 6, 7, 8, 9, 10, 11 or 12 species selected from the group.

4. The method of claim 3, wherein the dietary supplement supplies the deficient bacterial species.

5. A method for identifying the bacterial species of a genomic DNA sample, comprising:
sequencing hypervariable regions of 16S rRNA genes in the genomic DNA sample, and counting the copy number of each hypervariable region in the 16S rRNA genes in the genomic DNA sample; and
comparing the hypervariable region sequences and copy numbers thereof in the 16S rRNA genes to a database comprising hypervariable region sequences and copy numbers thereof in 16S rRNA genes of different bacterial species, to identify the bacterial species of the 16S rRNA genes.

6. The method of claim 5, further comprising pre-processing the hypervariable region sequences, wherein the pre-processing comprises one or more of de-noising, un-trimming, and abundance estimation.

7. The method of claim 6, further comprising trimming the sequences to a predetermined length, prior to un-trimming, which comprises concatenating the trimmed sequences.

8. The method of claim 7, further comprising aligning the sequences to the database.

9. The method of claim 8, wherein the database is prepared by a method comprising:
acquiring genome sequences from a plurality of bacterial species and strains;
extracting 16S rRNA sequences from the genome sequences;
eliminating sequences that are not within hypervariable regions of 16S rRNA genes;
counting sequence variants and copy numbers of unique hypervariable regions; and
identifying a list of 16S rRNA gene sequences from the bacterial species and strains.

10. The method of claim 9, wherein preparation of the database further comprises cleaning the 16S rRNA gene sequences in the list by removing outliers.

11. The method of claim 10, wherein preparation of the database further comprises cleaning the 16S rRNA gene sequences in the list by removing multi-annotated ones.

12. A method for preparing a database of 16S rRNA genes useful for identifying bacterial species, comprising:
acquiring genome sequences from a plurality of bacterial species and strains;
extracting 16S rRNA sequences from the genome sequences;
eliminating sequences that are not within hypervariable regions of 16S rRNA genes;
counting sequence variants and copy numbers of unique hypervariable regions; and
identifying a list of 16S rRNA gene sequences from the bacterial species and strains.

13. The method of claim 12, wherein preparation of the database further comprises cleaning the 16S rRNA gene sequences in the list by removing outliers.

14. The method of claim 13, wherein preparation of the database further comprises cleaning the 16S rRNA gene sequences in the list by removing multi-annotated ones.
